(19) United States
(12) Patent Application Publication
(10) Pub. No.: US 2010/0158801 A1
(43) Pub. Date: Jun. 24, 2010

(54) METHODS FOR THE TREATMENT OF ACUTE MYELOID LEUKEMIA

(76) Inventors: David S.F. Young, Toronto (CA); Daniel S. Pereira, Mississauga (CA); John E. Dick, Toronto (CA)

Correspondence Address: FISH & RICHARDSON PC, P.O. BOX 1022, MINNEAPOLIS, MN, 55440-1022, US

(21) Appl. No.: 12/342,986

(22) Filed: Dec. 23, 2008

(51) Int. Cl. A61K 51/00; A61K 39/395; G01N 33/574

(52) U.S. Cl. 424/1.49; 424/155.1; 424/178.1; 424/133.1; 435/7.23

(57) ABSTRACT

This invention relates to the staging, diagnosis and treatment of cancerous diseases (both primary tumors and tumor metastases), particularly to the mediation of cytotoxicity of tumor cells; and most particularly to the use of isolated monoclonal antibodies, antigen binding fragments thereof, and/or cancerous disease modifying antibodies (CDMAB), optionally in combination with one or more CDMAB, chemotherapeutic agents, and conjugates thereof, as a means for initiating a cytotoxic response to human hematologic malignancies. The invention further relates to binding assays, which utilize the isolated monoclonal antibodies, antigen binding fragments thereof, and/or CDMAB of the instant invention. The cancerous disease modifying antibodies antibodies can be conjugated to toxins, enzymes, radioactive compounds, cytokines, interferons, target or reporter moieties and hematogenous cells.

### FIELD OF THE INVENTION

This invention relates to the diagnosis and treatment of hematologic malignancies such as acute myeloid leukemia. More particularly, it is related to the use of cancerous disease modifying antibodies (CDMAB), optionally in combination with one or more CDMAB/chemotherapeutic agents, as a means for treating such diseases.

### BACKGROUND OF THE INVENTION

The cell membrane contains many different cell-surface proteins, some in motion and some anchored to the cytoskeleton. This huge repertoire of cell-surface proteins is capable of executing different functions such as signaling and adhesion. It is also known that certain types of membrane proteins are responsible for the organization of these cell-surface proteins into complexes capable of united functions that they could not carry out as single molecules. This emerging family of proteins, the tetraspanins or transmembrane 4 (TM4) family of integral membrane proteins, serves as a molecular facilitator or organizer of multi-molecular complexes.

Tetraspanins have been implicated in a large variety of physiological processes such as immune cell activation, cell migration, cell-cell fusion (including fertilization) and various aspects of cellular differentiation. These molecules have also been shown to play a role in infectious diseases (e.g. malaria, hepatitis C and human immunodeficiency virus) and several genetic diseases are linked to mutations in these molecules (e.g. X-linked mental retardation, retinal degeneration and incorrect assembly of human basement membranes in the kidney and skin) (Boucheix and Rubinstein. Cell. Mol. Life Sci. 58(9):1189-1205 2001). The ability of tetraspanins to interact with many other signaling molecules and participate in activation, adhesion and cell differentiation all relate to its role as “molecular facilitators” that bring together large molecular complexes and allow them, through stabilization, to function more efficiently. The interaction of tetraspanins with other signaling molecules is sometimes referred to as the tetraspanin web.

This super family (TM4SF) was first recognized in 1990, when comparison of the sequences of the newly cloned CD37, CD81 (TAPA-1) and sm23 genes with the tumor-associated gene CD63 (ME491) (Hotta et al. Cancer Res. 48(11):2955-2962 1988) revealed sequence homology and a conserved predicted structure (Wright et al. J Immunol 144(8):3195-3200 1990; Oren et al. Mol. Cell. Biol 10(8):4007-4015 1990). The family has now grown to about 32 members in humans (Le Naour et al. Proteomics. 6(24):6447-54 2006).

CD9 is a 24 kDa member of this family that is expressed on both hematopoietic and nonhematopoietic cells. Especially high concentrations of CD9 are expressed on the surface of platelets and endothelial cells (Forsyth K D. Immunology 72(2):292-296 1991; Jennings et al. Blood 88(10):624a 1996). CD9 was also recently discovered to be a member of the family of cell surface molecular complexes that include the integrins, other cell surface receptors and other tetraspanins. Several TM4 family members, including CD9, have been found to associate with β1 integrins as well as β2, β3, and β7 integrins (Rubinstein et al. Eur. J. Immunol. 24(12):3005-3013 1994; Nakamura et al. J. Cell Biol. 129(6):1691-1705 1995; Berditchevski et al. Mol. Biol. Cell. 7(2):193-207 1996; Radford et al. Biochem. Biophys. Res. Commun. 222(1):13-28 1996; Hadjiargyrou et al. J Neurochem 67(6):2505-2513 1996; Slupsky et al. Eur J Biochem 244(1):168-175 1997).

Based on cDNA sequence analysis, the TM4SF members are predicted to be single polypeptide chains with four highly hydrophobic putative transmembrane (TM) regions and two extracellular (EC) loops with both the amino and carboxy termini localized intracellularly. Alignment of all tetraspanin amino acid sequences revealed that much of the homology between tetraspanins is confined to the transmembrane domains, which contain a few highly conserved polar amino acids (an asparagine in TM1 and a glutamate or glutamine in TM3 and TM4). These charged residues within the membrane may interact with each other and may be important for the stability of protein assembly, as has been demonstrated for the T cell receptor (Cosson et al. Nature 351(6325):414-416 1991).

There are also conserved hydrophobic residues in all four transmembrane domains; some in TM2 are found in 17/18 tetraspanin sequences. The short region between TM2 and TM3 contains two to three charged residues, including a conserved glutamic acid. These homologies are not shared with other protein families that also have four transmembrane domains, such as the ligand-gated ion channels, connexins, or CD2O/FcERII3.

The conservation between residues observed in the putative transmembrane domains and certain residues in the EC loops, suggests that these proteins perform closely related functions (Maecker et al FASEB J 11(6):428-442 1997). There is greater sequence divergence in the extracellular loops of tetraspanins, although three cysteines in EC2 are located at defined distances from the TM regions in 16/18 family members. Two of these cysteines occur in a conserved CCG motif located about 50 amino acids past TM3. The third cysteine is often preceded by a glycine and is fixed at 11 amino acids upstream of TM4. A fourth conserved cysteine, frequently found in a PXSC motif, is variably placed in EC2. For some members of this family the use of reducing agents affects their recognition by antibodies indicating that disulfide bonding occurs. Which cysteines are involved is unknown but at least two of the conserved residues in the EC2 are implicated in disulfide bonding (Tomlinson et al. Eur J Immunol 23(1):136-140 1993).

Most of the tetraspanins are modified by N-glycosylation; some are variably glycosylated or acylated, such as CD9 (Seehafer et al. Biochim Biophys Acta 957(3):399-410 1988). The glycosylation patterns between different tetraspanins vary widely. CD9 contains a glycosylation site in EC1 (Boucheix et al. J Biol Chem 266(1):117-122 1991), whereas most other glycosylated tetraspanins contain sites in EC2 (Classon et al. J Exp Med 169(4):1497-1502). Within individual members, however, most glycosylation sites are conserved between species. For example, mouse, rat, primates and cow CD9 all have identical single glycosylation sites, whereas the feline molecule has lost this site altogether.

The expression pattern of some of these proteins have nearly ubiquitous tissue distribution (CD9, CD63, CD81, CD82) whereas others are highly restricted, for example, to lymphoid and myeloid cells (CD53) or mature B cells (CD37). Some members appear to be highly expressed in the immune system; more recently, their expression in the nervous system has also been appreciated. CD9 is transiently expressed in developing spinal motoneurons and other fetal central and peripheral nervous system sites (Tole and Patterson. Dev Dyn 197(2):94-106 1993). It is present in embryonic and fetal hematopoietic tissues (Abe et al Nippon Ketsucki Gakkai Zasshi. 1989 52(4):712-20 1989; Abe J. Clin Immunol Immunopathol. 1989 51(1):13-21 1989) and is also expressed during B cell development (Boucheix et al J Biol Chem 266(1):117-122 1991).

Interaction of CD9 with β1 integrins as well as β2, β3, and β7 integrins in particular, suggests that CD9 expression may influence many of the same cellular functions that have been assigned to the integrins. CD9 and other tetraspanins have been reported to participate in the activation, adhesion, and motility of cells as well as in normal and tumor cell growth (Maecker et al. FASEB J 11(6):428-442 1997). While it has been suggested that TM4 family members serve as molecular facilitators (Maecker et al. FASEB J 11(6):428-442 1997), their mode of influence may vary between cells. The transfection of CD9 into poorly motile CD9-negative pre-B cells (Raji) upregulated the motility of these cells across fibronectin and laminin (Shaw et al. 270(41):24092-24099 1995), while transfection of CD9 into nonlymphoid, motile cell lines downregulated their motility to these extracellular matrix components (Ikeyama et al. J. Exp. Med. 177, 1231-1237 1993).

Fibronectin was identified as a potential ligand for CD9 by demonstrating direct binding of fibronectin to immobilized platelet CD9 and to recombinant CD9 (Wilkinson et al. FASEB J. 9:A1500. 23 1995). By using mock-and CD9-transfected CHO cells, Cook et al., compared the adhesion and spreading of these transfected cells to immobilized extracellular matrix components, particularly fibronectin. They showed that: (i) the surface expression of CD9 modifies CHO cell adhesion and spread morphology on fibronectin, (ii) CD9 CHO cell-fibronectin interaction involves primarily the fibronectin segment composed of the HEP2/IIICS binding domain and (iii) CD9 expression down regulates the production of a pericellular fibronectin matrix. These data clearly suggested that ectopic CD9 expression may regulate cell-fibronectin interactions through CD9 binding to specific regions on fibronectin and through modulation of other fibronectin-binding molecules such as α5b1 (Cook et al. Exp Cell Res. 251(2):356-371).

While a number of the associations of tetraspanins are now reasonably well characterized in terms of physical and functional association, others remain controversial, particularly the association of tetraspanins and Fc receptors (FcR). After the demonstration that anti-CD9 antibodies trigger platelet aggregation, it was reported that the antibodies induce association of CD9 with the integrin αIIb/III (GPIIb/IIIa; CD4I/CD61) on platelets and that the triggering of platelet aggregation is mediated by GPIIb/IIIa (Slupsky et al. J Biol Chem. 264(21):12289-12293 1989). In fact, injection of anti-CD9 into monkeys causes lethal thrombocytopenia within 5 minutes of injection, which is prevented by pretreatment of the monkeys with anti-αIIb/β antibodies (Kawakatsu et al. Thromb Res. 70(3):245-254 1993). CD9-mediated platelet activation, like the activation induced by anti-αIIb/βIII antibodies, can be blocked by antibodies to FcγRII suggesting that the activation is mediated by FcγRII. Indeed, antibodies to several platelet proteins, including the tetraspanin PETA-3, induce platelet aggregation that is inhibited by Fc receptor blockade.

However, the vast majority of this data describes an indirect relationship because the cellular activation events result from co-ligation of tetraspanins with FcR via the Fc region of intact anti-tetraspanin antibodies. This event is unlikely to be of any significance in normal physiology. The fact that tetraspanins have so frequently been identified as the targets of antibodies which co-ligate FcR is suggestive of a spatial relationship between these molecules. The plethora of reports of tetraspanin-FcR co-ligation has perhaps drawn attention to more physiologically relevant reports which support this relationship, specifically showing proximal co-localization of tetraspanins with FcR by immuno fluorescence and co-immunoprecipitation (Higginbottom et al. 99(4):546-552 2000; Kaji et al. J Immunol 166(5):3256-3265 2001). Such interaction would facilitate cross-talk between FcR and adhesion/signaling molecules in the tetraspanin web which would have clear physiological significance to platelet and immune cell biology. That association of FcR with tetraspanins has important functional effects is implied by the demonstration of tetraspanin-dependent modulation of FcR signaling, both in co-ligation complexes and independently of co-ligation events.

In cancer, clinical studies have reported a link between tetraspanin expression levels and prognosis and/or metastasis. CD9 was initially described on the surface of cells of B-lineage acute lymphoblastic leukemia (Kersey et al. J Exp Med. 153(3):726-31 1981). It is expressed on 90 percent of B-lineage acute leukemias, and on 50 percent of acute myeloid leukemias and B-lineage chronic lymphoid leukemias (Boucheix et al. Leuk Res. 9(5):597-604 1985). In particular, CD9 is a constant marker of acute promyelocytic. The surface presence of CD9 may serve as a prognostic indicator of the metastatic potential of some cancers (Ikeyama et al. J Exp Med. 177(5):1231-1237 1993; Miyake et al. Cancer Res. 55(18):4127-4131 1995). Indeed a high level of the tetraspanins CD9 and CD82/KAI-1 on tumor cells is associated with a favorable prognosis in breast, lung, colon, prostate, and pancreatic cancers. Additionally, a decreased expression level of these molecules is correlated with metastasis in these cancers (Boucheix and Rubinstein. Cell Mol Life Sci. 58(9):1189-1205 2001). CD9 levels were often lower in cells obtained from lymph node metastases than in primary breast cancer tumor cells (Miyake et al. Cancer Res. 55(18):4127-4131 1995). Furthermore, using in vitro and in vivo experimental models, CD9 and CD82 have been shown to act as “metastasis suppressors” whereas CD151 was shown to increase the metastatic potential (Boucheix and Rubinstein. Cell Mol Life Sci. 58(9):1189-1205 2001).

Two recent proteomic studies of tetraspanin web composition in tumor and metastasis has been reported (Andre et al. Proteomics 6(5):1437-1449 2006; Le Naour et al. Mol Cell Proteomics 5(5):845-857 2006). These two reports were both focused on colon cancer using two different cellular models. The models were constituted of cell lines derived from primary colon tumors and metastases from the same patients. The first model was constituted by the cell lines SW480 (primary tumor) and SW620 (lymph node metastasis) (Leibovitz et al. Cancer Res 36(12):4562-4569 1976), available from the American Type Culture Collection (ATCC). The tetraspanin complexes were isolated after immunoaffinity purification and the proteins were identified by MS using LC-ESI-MS/MS and MALDI-FTICR.

The second model was constituted by the three cell lines Isrecol (IS1, primary tumor), Isreco2 (IS2, liver metastasis), and Isreco3 (IS3, peritoneal metastasis) (Cajot et al. J Biol Chem. 274(45):31903-31908 1997), established at the ISREC (Institut Suisse d'Etudes Experimentales sur le Cancer, Swiss). In this study, cells were lysed with the mild detergent Brij97 followed by immunoprecipitation experiments of the CD9-containing complexes. The associated proteins were further eluted using the more stringent detergent Triton X-100, which dissociates tetraspanin-tetraspanin associations. In order to rule out non-specific binding, immunoprecipitation experiments were also performed using an unrelated IgG1 that was treated identically to CD9 mAbs. Protein identification was performed by mass-spectrometry.

A comparative analysis of primary tumor cells and metastases in the two cellular models showed that some proteins were differentially detected. For most of these proteins, the differential expression was confirmed by quantitative methods such as flow cytometry. Important variations in the expression levels of several adhesion molecules were observed, in particular, receptors of the extracellular matrix such as laminin receptors. Interestingly, integrin α6b4 was detected by MS only in CD9-containing complexes from metastases. Immunoprecipitation and Western blotting experiments confirmed that a higher amount of integrin α6b4 was coimmunoprecipitated with CD9 in metastases from both models, despite a similar or lower expression level at the cell surface. Therefore, this suggests a specific recruitment of the integrin α6b4 into tetraspanin-enriched microdomains during tumor progression. In contrast, a significant decrease in other laminin receptors, such as integrin α3b1 and the Ig protein Lu/B-CAM (lutheran/B-cell adhesion molecule), was observed in metastatic cell lines from the two cellular models used as well as on various other metastatic cell lines (Andre et al. Proteomics 6(5):1437-1449 2006).

Another adhesion molecule identified by MS was epithelial cell adhesion molecule (EpCAM). This protein is expressed in many human epithelial tissues and overexpressed in the majority of epithelial carcinomas (Armstrong and Eck. Cancer Biol Ther. 2(4):320-326 2003). Interestingly, it has been demonstrated that EpCAM can associate directly with the tetraspanin CD9. Thus, a substantial colocalization of these two molecules in the normal colon has been observed, whereas the level of co localization was lower in primary tumors and metastases (Le Naour et al Mol Cell Proteomics 5(5):845-857 2006). Proteomics has also revealed the presence of different membrane proteases (i.e. CD26/dipeptidyl peptidase 4 (DPPIV) expressed only on some metastatic cells) as well as several signaling molecules in tetraspanin-enriched microdomains. These findings may shed a new light on the function of tetraspanins, suggesting that the microdomains may play a role as a platform for enzymatic activities and signal transduction.

In another proteomic study Gronborg et al., demonstrated the use of stable isotope labeling with amino acids in cell culture (SILAC) method to compare the secreted proteins (secretome) from pancreatic cancer-derived cells with that from non-neoplastic pancreatic ductal cells. They identified several proteins that have not been correlated previously with pancreatic cancer including perlecan (HSPG2), CD9 antigen, fibronectin receptor (integrin β1), and a novel cytokine designated as predicted osteoblast protein (FAM3C). Particularly CD9 was identified to be elevated in cancer versus normal by a ratio of 8. Because CD9 was not previously described to be elevated in pancreatic cancer they carried out validation studies by immunohistochemistry (IHC) using pancreatic cancer tissue microarrays (TMAs). CD9 was expressed in robust membranous distribution in 7 of 18 (39 percent) pancreatic cancers on the TMA with no expression seen in adjacent normal pancreatic parenchyma (Gronborg et al. Mol Cell Proteomics. 5(1):157-171). CD9 labeling demonstrated a pattern of apical luminal accentuation similar to the pattern they have reported previously for other secreted proteins in pancreatic cancers such as prostate stem cell antigen and mesothelin (Argani et al Clin Cancer Res 7(12):3862-3868 2001; Argani et al Cancer Res. 61(11):4320-4324 2001). In addition, labeling of intraluminal contents was often seen within neoplastic glandular structures, consistent with CD9 secretion.

The protein level quantitation data obtained by the SILAC method was compared with the mRNA data obtained by a DNA microarray experiment. CD9 antigen, which SILAC demonstrated to be differentially over expressed in the pancreatic cancer secretome and was confirmed as being over expressed at the protein level, was down-regulated 2-fold in Pancl versus HPDE cells based on DNA microarray data. This data reinforce the importance of assessing both the transcriptome and the proteome of human cancers (Gronborg et al. Mol Cell Proteomics. 5(1):157-171).

In another study, the expression of CD9 was examined in primary and metastatic gastric carcinoma tissues. In total, specimens from 78 patients were used for immunohistological staining and specimens from 57 patients were subjected to Northern blotting. CD9 expression was observed at both the message level and the protein level in primary gastric carcinoma tissues, lymph node metastatic tissues, and peritoneal dissemination tissues. CD9 expression was intensified in cancerous areas of gastric cancers in comparison with non cancerous areas in the same patient. When analyzed by the malignancy status based on the clinicopathological diagnosis, there was a tendency that CD9 expression was observed in severe vessel invasion, active lymph node metastasis, and advanced stage. These authors conclude that CD9 expression was rather intensified in gastric cancer tissue in comparison with normal tissues. CD9 expression was more prominent in advanced gastric cancer (Haruko et al. J Surg Res. 117(2):208-215 2004).

The role of CD9 in prostate carcinoma progression was also studied (Wang et al. Clin Cancer Res. 13(8):2354-2361 2007). Reduced or loss of CD9 expression within prostate neoplastic cells was observed in 24 percent of 107 clinically localized primary adenocarcinomas, 85 percent of 60 clinically advanced primary adenocarcinomas, 85 percent of 65 lymph node metastases and 65 percent of 23 bone metastases. This reduction in CD9 expression was associated to alterations of CD9 cDNA not observed in normal tissues. They found that all PC-3 derived cell lines, one PIN and four prostatic adenocarcinomas harbored deletions in their CD9 cDNAs. These deletions removed nucleotides 115 to 487, 190 to 585 or 120 to 619 of the 684 bp CD9 coding sequence. Thus, from the 228 amino acid CD9 protein, amino acids 39 to 163, 64 to 195 or 40 to 207 were eliminated by these deletions. These deletions affected the large extracellular and intracellular domains of the protein. The presence of the PC-3M-LN4 deletion (deletion 64-195) was confirmed on direct sequencing of the mRNA amplification product (without cloning). These deletions were not detected in genomic DNA derived from some of these samples, arguing for the existence of transcriptional CD9 mRNA modifications. Another deletion was detected in the DU145 cell line, whereas an in-frame insertion was present in mRNA derived from PC-3M-Pro4.

Lastly, common missense point mutations were observed in one prostatic carcinoma cell line (PC-3M-LN4), one specimen of PIN, and seven specimens of prostatic adenocarcinoma. Some specimens were harboring more than one missense mutation. Interestingly, CD9 protein expression was not detected in most of these cases (except in one specimen of prostatic adenocarcinoma). A base pair substitution resulting in a new stop codon, located in the second cytoplasmic domain (amino acid 83), was also present in one PIN and in two prostate cancer patients where they did not detect the CD9 protein. Although reduced expression of CD9 protein has been associated with cancer progression in different tumor types, this is the first report implicating CD9 mRNA alterations in CD9 protein inactivation.

The role of CD9 in several cell lines has also been investigated by using anti-CD9 monoclonal antibodies. These experiments demonstrated effects in adhesion and proliferation depending on the cell type and the antibody used. Anti-CD9 antibodies stimulated fibrin clot retraction by fibroblasts (Azzarone et al. J Cell Physiol. 125(3):420-426 1985), induced homotypic adhesion in pre-B lymphocytes (Masellis-Smith et al. J Immunol. 144(5):1607-1613 1990), inhibited the motility of lung adenocarcinoma cells (Miyake et al. J Exp Med. 174(6):1347-1354 1991), augmented the adherence of neutrophils to endothelial cells (Forsyth K D. Immunology 72(2):292-296 1991) and elicited phosphatidylinositol turnover, phosphatidylinositol biosynthesis and protein-tyrosine phosphorylation in human platelets (Yatomi et al. FEBS Lett. 322(3):285-290 1993). One anti-CD9 monoclonal antibody, B2C11, promoted adhesion of a number of Schwann cell lines, PC12 cells and primary rat Schwann cells (Hadjiargyrou and Patterson. J Neurosci. 15(1 Pt 2):574-583 1995). In addition, this antibody also stimulated proliferation of one of the Schwann cell lines. In another article the same group further demonstrated that another anti-CD9 monoclonal antibody, SMRA1, enhanced motility and migration in primary Schwann cells which is correlated with an increase in cytosolic calcium and phosphoproteins (Anton et al. J Neurosci. 15(1 Pt 2):584-95 1994). However, none of these antibodies have been reported to have been tested in an in vivo model of human cancer.

Finally, a recent report showed that ectopic expression of CD9 in colon carcinoma cells resulted in enhanced integrin-dependent adhesion and inhibition of cell growth. Consistent with these effects, treatment of these cells with anti-CD9 specific antibodies resulted in (i) increased β1 integrin-mediated cell adhesion through a mechanism involving clustering of integrin molecules rather than altered affinity; (ii) induction of morphological changes characterized by the acquisition of an elongated cell phenotype; (iii) inhibition of cell proliferation with no significant effect on cell survival; (iv) increased expression of membrane TNF-α and finally (v) inhibition of the in vivo tumorigenic capacity in nude mice. In addition, through the use of selective blockers of TNF-α, they have demonstrated that this cytokine partly mediates the anti-proliferative effects of CD9 (Ovalle et al. Int J Cancer. [Epub ahead of print] 2007). The two anti-CD9 antibodies tested in vivo, VJ1/20 and PAINS-13, were tested in a prophylactic type xenograft model whereas the anti-CD9 antibodies disclosed herein have demonstrated efficacy in both prophylactic and, more clinically relevant, established xenograft models of human cancer. In addition, unlike VJ1/20 or PAINS-13, the anti-CD9 antibodies disclosed herein have demonstrated in vivo efficacy in more than one cancer xenograft model.

Acute myeloid leukemia (AML) is a heterogeneous clonal disorder marked by accumulation of undifferentiated myeloid blasts. The leukemic hierarchy is continuously replenished by rare, functionally distinct Leukemic stem cells (LSCs) characterized by their capacity for self-renewal as well as the ability to generate clonogenic leukemic progenitors that ultimately produce large numbers of leukemic blasts. Many studies have reported that AML LSCs are quiescent or slowly dividing, whereas clonogenic progenitors are rapidly proliferating. LSCs in AML have been found to be the only cells that initiate and maintain the hierarchy of leukemic clones and therefore contribute to leukemia progression and relapse. This finding suggests that a permanent cure for AML requires elimination of LSCs. AML LSCs are homologous to normal HSCs in many ways, including a CD34+CD38− phenotype, but they show enhanced self-renewal and some discordant expression of cell surface markers.

One strategy for the development of LSC targeted therapies is to target cell surface antigens with activities required for LSC function. Current antiproliferative chemotherapeutic regimens typically target these rapidly cycling progenitors, inducing disease remission. Relapse is frequent, however, and <30% of adults with AML survive for long, suggesting that quiescent AML LSCs are not effectively eradicated by current therapies. Thus, there is great need for new therapies that eliminate AML LSCs by targeting their specific properties while leaving normal hematopoietic stem cells (HSCs) virtually unharmed.

Monoclonal Antibodies as Cancer Therapy: Each individual who presents with cancer is unique and has a cancer that is as different from other cancers as that person's identity. Despite this, current therapy treats all patients with the same type of cancer, at the same stage, in the same way. At least 30 percent of these patients will fail the first line therapy, thus leading to further rounds of treatment and the increased probability of treatment failure, metastases, and ultimately, death. A superior approach to treatment would be the customization of therapy for the particular individual. The only current therapy which lends itself to customization is surgery. Chemotherapy and radiation treatment cannot be tailored to the patient, and surgery by itself, in most cases is inadequate for producing cures.

With the advent of monoclonal antibodies, the possibility of developing methods for customized therapy became more realistic since each antibody can be directed to a single epitope. Furthermore, it is possible to produce a combination of antibodies that are directed to the constellation of epitopes that uniquely define a particular individual's tumor.

Having recognized that a significant difference between cancerous and normal cells is that cancerous cells contain antigens that are specific to transformed cells, the scientific community has long held that monoclonal antibodies can be designed to specifically target transformed cells by binding specifically to these cancer antigens; thus giving rise to the belief that monoclonal antibodies can serve as “Magic Bullets” to eliminate cancer cells. However, it is now widely recognized that no single monoclonal antibody can serve in all instances of cancer, and that monoclonal antibodies can be deployed, as a class, as targeted cancer treatments. Monoclonal antibodies isolated in accordance with the teachings of the instantly disclosed invention have been shown to modify the cancerous disease process in a manner which is beneficial to the patient, for example by reducing the tumor burden, and will variously be referred to herein as cancerous disease modifying antibodies (CDMAB) or “anti-cancer” antibodies.

At the present time, the cancer patient usually has few options of treatment. The regimented approach to cancer therapy has produced improvements in global survival and morbidity rates. However, to the particular individual, these improved statistics do not necessarily correlate with an improvement in their personal situation.

Thus, if a methodology was put forth which enabled the practitioner to treat each tumor independently of other patients in the same cohort, this would permit the unique approach of tailoring therapy to just that one person. Such a course of therapy would, ideally, increase the rate of cures, and produce better outcomes, thereby satisfying a long-felt need.

Historically, the use of polyclonal antibodies has been used with limited success in the treatment of human cancers. Lymphomas and leukemias have been treated with human plasma, but there were few prolonged remission or responses. Furthermore, there was a lack of reproducibility and there was no additional benefit compared to chemotherapy. Solid tumors such as breast cancers, melanomas and renal cell carcinomas have also been treated with human blood, chimpanzee serum, human plasma and horse serum with correspondingly unpredictable and ineffective results.

There have been many clinical trials of monoclonal antibodies for solid tumors. In the 1980s there were at least four clinical trials for human breast cancer which produced only one responder from at least 47 patients using antibodies against specific antigens or based on tissue selectivity. It was not until 1998 that there was a successful clinical trial using a humanized anti-Her2/neu antibody (Herceptin®) in combination with CISPLATIN. In this trial 37 patients were assessed for responses of which about a quarter had a partial response rate and an additional quarter had minor or stable disease progression. The median time to progression among the responders was 8.4 months with median response duration of 5.3 months.

Herceptin® was approved in 1998 for first line use in combination with Taxol®. Clinical study results showed an increase in the median time to disease progression for those who received antibody therapy plus Taxol® (6.9 months) in comparison to the group that received Taxol® alone (3.0 months). There was also a slight increase in median survival; 22 versus 18 months for the Herceptin® plus Taxol® treatment arm versus the Taxol® treatment alone arm. In addition, there was an increase in the number of both complete (8 versus 2 percent) and partial responders (34 versus 15 percent) in the antibody plus Taxol® combination group in comparison to Taxol® alone. However, treatment with Herceptin® and Taxol® led to a higher incidence of cardiotoxicity in comparison to Taxol® treatment alone (13 versus 1 percent respectively). Also, Herceptin® therapy was only effective for patients who over express (as determined through immunohistochemistry (IHC) analysis) the human epidermal growth factor receptor 2 (Her2/neu), a receptor, which currently has no known function or biologically important ligand; approximately 25 percent of patients who have metastatic breast cancer. Therefore, there is still a large unmet need for patients with breast cancer. Even those who can benefit from Herceptin® treatment would still require chemotherapy and consequently would still have to deal with, at least to some degree, the side effects of this kind of treatment.

The clinical trials investigating colorectal cancer involve antibodies against both glycoprotein and glycolipid targets. Antibodies such as 17-1A, which has some specificity for adenocarcinomas, has undergone Phase 2 clinical trials in over 60 patients with only 1 patient having a partial response. In other trials, use of 17-1A produced only 1 complete response and 2 minor responses among 52 patients in protocols using additional cyclophosphamide. To date, Phase III clinical trials of 17-1A have not demonstrated improved efficacy as adjuvant therapy for stage III colon cancer. The use of a humanized murine monoclonal antibody initially approved for imaging also did not produce tumor regression.

Only recently have there been any positive results from colorectal cancer clinical studies with the use of monoclonal antibodies. In 2004, ERBITUX® was approved for the second line treatment of patients with EGFR-expressing metastatic colorectal cancer who are refractory to irinotecan-based chemotherapy. Results from both a two-arm Phase II clinical study and a single arm study showed that ERBITUX® in combination with irinotecan had a response rate of 23 and 15 percent respectively with a median time to disease progression of 4.1 and 6.5 months respectively. Results from the same two-arm Phase II clinical study and another single arm study showed that treatment with ERBITUX® alone resulted in an 11 and 9 percent response rate respectively with a median time to disease progression of 1.5 and 4.2 months respectively.

Consequently in both Switzerland and the United States, ERBITUX® treatment in combination with irinotecan, and in the United States, ERBITUX® treatment alone, has been approved as a second line treatment of colon cancer patients who have failed first line irinotecan therapy. Therefore, like Herceptin®, treatment in Switzerland is only approved as a combination of monoclonal antibody and chemotherapy. In addition, treatment in both Switzerland and the US is only approved for patients as a second line therapy. Also, in 2004, AVASTIN® was approved for use in combination with intravenous 5-fluorouracil-based chemotherapy as a first line treatment of metastatic colorectal cancer. Phase III clinical study results demonstrated a prolongation in the median survival of patients treated with AVASTIN® plus 5-fluorouracil compared to patients treated with 5-fluourouracil alone (20 months versus 16 months respectively). However, again like Herceptin® and ERBITUX®, treatment is only approved as a combination of monoclonal antibody and chemotherapy.

There also continues to be poor results for lung, brain, ovarian, pancreatic, prostate, and stomach cancer. The most promising recent results for non-small cell lung cancer came from a Phase II clinical trial where treatment involved a monoclonal antibody (SGN-15; dox-BR96, anti-Sialyl-LeX) conjugated to the cell-killing drug doxorubicin in combination with the chemotherapeutic agent TAXOTERE®. TAXOTERE® is the only FDA approved chemotherapy for the second line treatment of lung cancer. Initial data indicate an improved overall survival compared to TAXOTERE® alone. Out of the 62 patients who were recruited for the study, two-thirds received SGN-15 in combination with TAXOTERE® while the remaining one-third received TAXOTERE® alone. For the patients receiving SGN-15 in combination with TAXOTERE®, median overall survival was 7.3 months in comparison to 5.9 months for patients receiving TAXOTERE® alone. Overall survival at 1 year and 18 months was 29 and 18 percent respectively for patients receiving SNG-15 plus TAXOTERE® compared to 24 and 8 percent respectively for patients receiving TAXOTERE® alone. Further clinical trials are planned.

Preclinically, there has been some limited success in the use of monoclonal antibodies for melanoma. Very few of these antibodies have reached clinical trials and to date none have been approved or demonstrated favorable results in Phase III clinical trials.

The discovery of new drugs to treat disease is hindered by the lack of identification of relevant targets among the products of 30,000 known genes that could contribute to disease pathogenesis. In oncology research, potential drug targets are often selected simply due to the fact that they are over-expressed in tumor cells. Targets thus identified are then screened for interaction with a multitude of compounds. In the case of potential antibody therapies, these candidate compounds are usually derived from traditional methods of monoclonal antibody generation according to the fundamental principles laid down by Kohler and Milstein (1975, Nature, 256, 495-497, Kohler and Milstein). Spleen cells are collected from mice immunized with antigen (e.g. whole cells, cell fractions, purified antigen) and fused with immortalized hybridoma partners. The resulting hybridomas are screened and selected for secretion of antibodies which bind most avidly to the target. Many therapeutic and diagnostic antibodies directed against cancer cells, including Herceptin® and RITUXIMAB, have been produced using these methods and selected on the basis of their affinity. The flaws in this strategy are two-fold. Firstly, the choice of appropriate targets for therapeutic or diagnostic antibody binding is limited by the paucity of knowledge surrounding tissue specific carcinogenic processes and the resulting simplistic methods, such as selection by overexpression, by which these targets are identified. Secondly, the assumption that the drug molecule that binds to the receptor with the greatest affinity usually has the highest probability for initiating or inhibiting a signal may not always be the case.

Despite advances in the treatment against acute myeloid leukemia (AML), the long term-survival of AML patients is still poor due to disease relapse and the resistance to chemotherapy. Leukemic stem cells (LSCs) in AML have been found to be the only cells that initiate and maintain the hierarchy of leukemic clones and therefore contribute to leukemia progression and relapse. This finding suggests that a permanent cure for AML requires elimination of LSCs. One strategy for the development of LSC targeted therapies is to target cell surface antigens with activities required for LSC function.

### SUMMARY OF THE INVENTION

This application utilizes methodology for producing patient specific anti-cancer antibodies taught in the U.S. Pat. No. 6,180,357 patent for isolating hybridoma cell lines which encode for cancerous disease modifying monoclonal antibodies. These antibodies can be made specifically for one tumor and thus make possible the customization of cancer therapy. Within the context of this application, anti-cancer antibodies having either cell-killing (cytotoxic) or cell-growth inhibiting (cytostatic) properties will hereafter be referred to as cytotoxic. These antibodies can be used in aid of staging and diagnosis of a cancer, and can be used to treat tumor metastases. These antibodies can also be used for the prevention of cancer by way of prophylactic treatment. Unlike antibodies generated according to traditional drug discovery paradigms, antibodies generated in this way may target molecules and pathways not previously shown to be integral to the growth and/or survival of malignant tissue. Furthermore, the binding affinities of these antibodies are suited to requirements for initiation of the cytotoxic events that may not be amenable to stronger affinity interactions. Also, it is within the purview of this invention to conjugate standard chemotherapeutic modalities, e.g. radionuclides, with the CDMAB of the instant invention, thereby focusing the use of said chemotherapeutics. The CDMAB can also be conjugated to toxins, cytotoxic moieties, enzymes e.g. biotin conjugated enzymes, cytokines, interferons, target or reporter moieties or hematogenous cells, thereby forming an antibody conjugate. The CDMAB can be used alone or in combination with one or more CDMAB/chemotherapeutic agents.

The prospect of individualized anti-cancer treatment will bring about a change in the way a patient is managed. A likely clinical scenario is that a tumor sample is obtained at the time of presentation, and banked. From this sample, the tumor can be typed from a panel of pre-existing cancerous disease modifying antibodies. The patient will be conventionally staged but the available antibodies can be of use in further staging the patient. The patient can be treated immediately with the existing antibodies, and a panel of antibodies specific to the tumor can be produced either using the methods outlined herein or through the use of phage display libraries in conjunction with the screening methods herein disclosed. All the antibodies generated will be added to the library of anti-cancer antibodies since there is a possibility that other tumors can bear some of the same epitopes as the one that is being treated. The antibodies produced according to this method may be useful to treat cancerous disease in any number of patients who have cancers that bind to these antibodies.

In addition to anti-cancer antibodies, the patient can elect to receive the currently recommended therapies as part of a multi-modal regimen of treatment. The fact that the antibodies isolated via the present methodology are relatively non-toxic to non-cancerous cells allows for combinations of antibodies at high doses to be used, either alone, or in conjunction with conventional therapy. The high therapeutic index will also permit re-treatment on a short time scale that should decrease the likelihood of emergence of treatment resistant cells. If the patient is refractory to the initial course of therapy or metastases develop, the process of generating specific antibodies to the tumor can be repeated for re-treatment. Furthermore, the anti-cancer antibodies can be conjugated to red blood cells obtained from that patient and re-infused for treatment of metastases. There have been few effective treatments for metastatic cancer and metastases usually portend a poor outcome resulting in death. However, metastatic cancers are usually well vascularized and the delivery of anti-cancer antibodies by red blood cells can have the effect of concentrating the antibodies at the site of the tumor. Even prior to metastases, most cancer cells are dependent on the host's blood supply for their survival and an anti-cancer antibody conjugated to red blood cells can be effective against in situ tumors as well. Alternatively, the antibodies may be conjugated to other hematogenous cells, e.g. lymphocytes, macrophages, monocytes, natural killer cells, etc.

There are five classes of antibodies and each is associated with a function that is conferred by its heavy chain. It is generally thought that cancer cell killing by naked antibodies are mediated either through antibody dependent cellular cytotoxicity (ADCC) or complement dependent cytotoxicity (CDC). For example murine IgM and IgG2a antibodies can activate human complement by binding the C-1 component of the complement system thereby activating the classical pathway of complement activation which can lead to tumor lysis. For human antibodies the most effective complement activating antibodies are generally IgM and IgG1. Murine antibodies of the IgG2a and IgG3 isotype are effective at recruiting cytotoxic cells that have Fc receptors which will lead to cell killing by monocytes, macrophages, granulocytes and certain lymphocytes. Human antibodies of both the IgG1 and IgG3 isotype mediate ADCC.

The cytotoxicity mediated through the Fc region requires the presence of effector cells, their corresponding receptors, or proteins e.g. NK cells, T-cells and complement. In the absence of these effector mechanisms, the Fc portion of an antibody is inert. The Fc portion of an antibody may confer properties that affect the pharmacokinetics of an antibody in vivo, but in vitro this is not operative.

The cytotoxicity assays under which we test the antibodies do not have any of the effector mechanisms present, and are carried out in vitro. These assays do not have effector cells (NK, Macrophages, or T-cells) or complement present. Since these assays are completely defined by what is added together, each component can be characterized. The assays used herein contain only target cells, media and sera. The target cells do not have effector functions since they are cancer cells or fibroblasts. Without exogenous cells which have effector function properties there is no cellular elements that have this function. The media does not contain complement or any cells. The sera used to support the growth of the target cells do not have complement activity as disclosed by the vendors. Furthermore, in our own labs we have verified the absence of complement activity in the sera used. Therefore, our work evidences the fact that the effects of the antibodies are due entirely to the effects of the antigen binding which is mediated through the Fab. Effectively, the target cells are seeing and interacting with only the Fab, since they do not have receptors for the Fc. Although, the hybridoma is secreting complete immunoglobulin which was tested with the target cells, the only part of the immunoglobulin that interacts with the cells are the Fab, which act as antigen binding fragments.

With respect to the instantly claimed antibodies and antigen binding fragments, the application, as filed, has demonstrated cellular cytotoxicity as evidenced by the data in FIG. 1. As pointed out above, and as herein confirmed via objective evidence, this effect was entirely due to binding by the Fab to the tumor cells.

Ample evidence exists in the art of antibodies mediating cytotoxicity due to direct binding of the antibody to the target antigen independent of effector mechanisms recruited by the Fc. The best evidence for this is in vitro experiments which do not have supplemental cells, or complement (to formally exclude those mechanisms). These types of experiments have been carried out with complete immunoglobulin, or with antigen binding fragments such as F(ab′)₂ fragments. In these types of experiments, antibodies or antigen binding fragments can directly induce apoptosis of target cells such as in the case of anti-Her2 and anti-EGFR antibodies, both of which have antibodies that are approved by the US FDA for marketing in cancer therapy.

Another possible mechanism of antibody mediated cancer killing may be through the use of antibodies that function to catalyze the hydrolysis of various chemical bonds in the cell membrane and its associated glycoproteins or glycolipids, so-called catalytic antibodies.

There are three additional mechanisms of antibody-mediated cancer cell killing. The first is the use of antibodies as a vaccine to induce the body to produce an immune response against the putative antigen that resides on the cancer cell. The second is the use of antibodies to target growth receptors and interfere with their function or to down regulate that receptor so that its function is effectively lost. The third is the effect of such antibodies on direct ligation of cell surface moieties that may lead to direct cell death, such as ligation of death receptors such as TRAIL R1 or TRAIL R2, or integrin molecules such as alpha V beta 3 and the like.

The clinical utility of a cancer drug is based on the benefit of the drug under an acceptable risk profile to the patient. In cancer therapy survival has generally been the most sought after benefit, however there are a number of other well-recognized benefits in addition to prolonging life. These other benefits, where treatment does not adversely affect survival, include symptom palliation, protection against adverse events, prolongation in time to recurrence or disease-free survival, and prolongation in time to progression. These criteria are generally accepted and regulatory bodies such as the U.S. Food and Drug Administration (F.D.A.) approve drugs that produce these benefits (Hirschfeld et al. Critical Reviews in Oncology/Hematology 42:137-143 2002). In addition to these criteria it is well recognized that there are other endpoints that may presage these types of benefits. In part, the accelerated approval process granted by the U.S. F.D.A. acknowledges that there are surrogates that will likely predict patient benefit. As of year-end 2003, there have been sixteen drugs approved under this process, and of these, four have gone on to full approval, i.e., follow-up studies have demonstrated direct patient benefit as predicted by surrogate endpoints. One important endpoint for determining drug effects in solid tumors is the assessment of tumor burden by measuring response to treatment (Therasse et al. Journal of the National Cancer Institute 92(3):205-216 2000). The clinical criteria (RECIST criteria) for such evaluation have been promulgated by Response Evaluation Criteria in Solid Tumors Working Group, a group of international experts in cancer. Drugs with a demonstrated effect on tumor burden, as shown by objective responses according to RECIST criteria, in comparison to the appropriate control group tend to, ultimately, produce direct patient benefit. In the pre-clinical setting tumor burden is generally more straightforward to assess and document. In that pre-clinical studies can be translated to the clinical setting, drugs that produce prolonged survival in pre-clinical models have the greatest anticipated clinical utility. Analogous to producing positive responses to clinical treatment, drugs that reduce tumor burden in the pre-clinical setting may also have significant direct impact on the disease. Although prolongation of survival is the most sought after clinical outcome from cancer drug treatment, there are other benefits that have clinical utility and it is clear that tumor burden reduction, which may correlate to a delay in disease progression, extended survival or both, can also lead to direct benefits and have clinical impact (Eckhardt et al. Developmental Therapeutics: Successes and Failures of Clinical Trial Designs of Targeted Compounds; ASCO Educational Book, 39^{th} Annual Meeting, 2003, pages 209-219).

The instant inventors have previously discovered that anti-CD9 monoclonal antibody AR40A746.2.3, is useful in a cytotoxic assay and in an animal model of solid human cancers. Furthermore, the AR40A746.2.3 antigen is a target for a therapeutic agent, that when administered can reduce the tumor burden of a solid human cancer expressing the antigen in a mammal. The use of CDMAB AR40A746.2.3 and its derivatives, and antigen binding fragments thereof, and cytotoxicity inducing ligands thereof, to target their antigen to reduce the tumor burden of a cancer expressing CD9 in a mammal has previously been demonstrated, as has the use of AR40A746.2.3 for detecting CD9 in cancerous cells that can be useful for the diagnosis, prediction of therapy, and prognosis of mammals bearing tumors that express this antigen.

It has now been discovered that CD9 is differentially overexpressed in CD34+CD38− leukemic stem cells versus CD34+CD38− normal stem cells. It has also been shown that treatment with AR40A746.2.3 in an in vivo model of AML inhibits leukemic outgrowth in test animals, and that this effect has occurred via inhibition of the human AML cancer stem cell which led to the outgrowth.

It is an objective of the invention to teach a method of treatment of hematologic malignancies such as AML using CDMAB AR40A746.2.3.

It is another objective of the invention to teach the diagnosis of hematologic malignancies such as AML using CDMAB AR40A746.2.3.

It is a further objective of the invention to teach the use of CDMAB AR40A746.2.3 for the prognosis or staging of hematologic malignancies in mammals.

It is a further objective of the invention to teach the use of CDMAB AR40A746.2.3 for the selection of a patient for the treatment of a hematologic malignancy.

Other objects and advantages of this invention will become apparent from the following description wherein are set forth, by way of illustration and example, certain embodiments of this invention.

### BRIEF DESCRIPTION OF THE FIGURES

FIG. 1 includes representative FACS histograms of AR40A746.2.3 antibodies directed against human CD34+CD38− leukemic and normal cells.

FIG. 2 compares the percentage of CD9 expressing human CD34+CD38− AML, ALL and CML leukemic and normal cord blood and adult bone marrow samples.

FIG. 3 demonstrates the effect of AR40A746.2.3 on human AML outgrowth in a murine model. The horizontal lines indicate the average percentage of human AML cells in the NOD/SCID bone marrow. Data points represent single mouse measurements.

FIG. 4 demonstrates the effect of AR40A746.2.3 on human AML outgrowth in secondary recipients. The horizontal lines indicate the average percentage of human AML cells in the NOD/SCID bone marrow. Data points represent single mouse measurements.

### DETAILED DESCRIPTION OF THE INVENTION

In general, the following words or phrases have the indicated definition when used in the summary, description, examples, and claims.

The term “antibody” is used in the broadest sense and specifically covers, for example, single monoclonal antibodies (including agonist, antagonist, and neutralizing antibodies, de-immunized, murine, chimeric or humanized antibodies), antibody compositions with polyepitopic specificity, single-chain antibodies, diabodies, triabodies, immunoconjugates and antibody fragments (see below).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma (murine or human) method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

“Antibody fragments” comprise a portion of an intact antibody, preferably comprising the antigen-binding or variable region thereof. Examples of antibody fragments include less than full length antibodies, Fab, Fab′, F(ab′)₂, and Fv fragments; diabodies; linear antibodies; single-chain antibody molecules; single-chain antibodies, single domain antibody molecules, fusion proteins, recombinant proteins and multispecific antibodies formed from antibody fragment(s).

An “intact” antibody is one which comprises an antigen-binding variable region as well as a light chain constant domain (CL) and heavy chain constant domains, C_{H}1, C_{H}2 and C_{H}3. The constant domains may be native sequence constant domains (e.g. human native sequence constant domains) or amino acid sequence variant thereof. Preferably, the intact antibody has one or more effector functions.

Depending on the amino acid sequence of the constant domain of their heavy chains, intact antibodies can be assigned to different “classes”. There are five-major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

Antibody “effector functions” refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs as described herein.

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daëron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FCR” herein. The term also includes the neonatal receptor, FcRn, which is responsible for the transfer of maternal IgGs to the fetus (Guyer et al., J. Immunol. 117:587 (1976) and Kim et al., Eur. J. Immunol 24:2429 (1994)).

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods 202:163 (1996), may be performed.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 2632 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (κ) and lambda (λ), based on the amino acid sequences of their constant domains.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a variable heavy domain (V_{H}) connected to a variable light domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al, Proc. Natl. Acad. Sci. USA, 90:6444-6448 (1993).

The term “triabodies” or “trivalent trimers” refers to the combination of three single chain antibodies. Triabodies are constructed with the amino acid terminus of a V_{L} or V_{H} domain, i.e., without any linker sequence. A triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. Isolated antibody includes the antibody in situ within recombinant cells since at least one component of the antibody's natural environment will not be present. Ordinarily, however, isolated antibody will be prepared by at least one purification step.

An antibody “which binds” an antigen of interest, e.g. CD9 antigen, is one capable of binding that antigen with sufficient affinity such that the antibody is useful as a therapeutic or diagnostic agent in targeting a cell expressing the antigen. Where the antibody is one which binds CD9, it will usually preferentially bind CD9 as opposed to other receptors, and does not include incidental binding such as non-specific Fc contact, or binding to post-translational modifications common to other antigens and may be one which does not significantly cross-react with other proteins. Methods, for the detection of an antibody that binds an antigen of interest, are well known in the art and can include but are not limited to assays such as FACS, cell ELISA and Western blot.

As used herein, the expressions “cell”, “cell line”, and “cell culture” are used interchangeably, and all such designations include progeny. It is also understood that all progeny may not be precisely identical in DNA content, due to deliberate or inadvertent mutations. Mutant progeny that have the same function or biological activity as screened for in the originally transformed cell are included. It will be clear from the context where distinct designations are intended.

“Treatment or treating” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. Hence, the mammal to be treated herein may have been diagnosed as having the disorder or may be predisposed or susceptible to the disorder.

The terms “cancer” and “cancerous” refer to or describe the physiological condition in mammals that is typically characterized by unregulated cell growth or death. Examples of cancer include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

“Hematologic malignancies” include, but are not limited to, diseases such as Acute Myeloid Leukemia (AML), Chronic Lymphocyte Leukemia (CLL), Chronic Myeloid Leukemia (CML) and Multiple Myeloma.

A “chemotherapeutic agent” is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carnomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiarniprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidarnol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Aventis, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

“Mammal” for purposes of treatment refers to any animal classified as a mammal, including humans, mice, SCID or nude mice or strains of mice, domestic and farm animals, and zoo, sports, or pet animals, such as sheep, dogs, horses, cats, cows, etc. Preferably, the mammal herein is human.

“Oligonucleotides” are short-length, single- or double-stranded polydeoxynucleotides that are chemically synthesized by known methods (such as phosphotriester, phosphite, or phosphoramidite chemistry, using solid phase techniques such as described in EP 266,032, published 4 May 1988, or via deoxynucleoside H-phosphonate intermediates as described by Froehler et al., Nucl. Acids Res., 14:5399-5407, 1986. They are then purified on polyacrylamide gels.

In accordance with the present invention, “humanized” and/or “chimeric” forms of non-human (e.g. murine) immunoglobulins refer to antibodies which contain specific chimeric immunoglobulins, immunoglobulin chains or fragments thereof (such as Fv, Fab, Fab′, F(ab′)₂ or other antigen-binding subsequences of antibodies) which results in the decrease of a human anti-mouse antibody (HAMA), human anti-chimeric antibody (HACA) or a human anti-human antibody (HAHA) response, compared to the original antibody, and contain the requisite portions (e.g. CDR(s), antigen binding region(s), variable domain(s) and so on) derived from said non-human immunoglobulin, necessary to reproduce the desired effect, while simultaneously retaining binding characteristics which are comparable to said non-human immunoglobulin. For the most part, humanized antibodies are human immunoglobulins (recipient antibody) in which residues from the complementarity determining regions (CDRs) of the recipient antibody are replaced by residues from the CDRs of a non-human species (donor antibody) such as mouse, rat or rabbit having the desired specificity, affinity and capacity. In some instances, Fv framework region (FR) residues of the human immunoglobulin are replaced by corresponding non-human FR residues. Furthermore, the humanized antibody may comprise residues which are found neither in the recipient antibody nor in the imported CDR or FR sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR residues are those of a human immunoglobulin consensus sequence. The humanized antibody optimally also will comprise at least a portion of an immunoglobulin constant region (Fc), typically that of a human immunoglobulin.

“De-immunized” antibodies are immunoglobulins that are non-immunogenic, or less immunogenic, to a given species. De-immunization can be achieved through structural alterations to the antibody. Any de-immunization technique known to those skilled in the art can be employed. One suitable technique for de-immunizing antibodies is described, for example, in WO 00/34317 published Jun. 15, 2000.

An antibody which induces “apoptosis” is one which induces programmed cell death by any means, illustrated by but not limited to binding of annexin V, caspase activity, fragmentation of DNA, cell shrinkage, dilation of endoplasmic reticulum, cell fragmentation, and/or formation of membrane vesicles (called apoptotic bodies).

As used herein “antibody induced cytotoxicity” is understood to mean the cytotoxic effect derived from the hybridoma supernatant or antibody produced by the hybridoma deposited with the IDAC as accession number 141204-01, a humanized antibody of the isolated monoclonal antibody produced by the hybridoma deposited with the IDAC as accession number 141204-01, a chimeric antibody of the isolated monoclonal antibody produced by the hybridoma deposited with the IDAC as accession number 141204-01, antigen binding fragments, or antibody ligands thereof, which effect is not necessarily related to the degree of binding.

The hybridoma cell line AR40A746.2.3 was deposited, in accordance with the Budapest Treaty, with the International Depository Authority of Canada (IDAC), Bureau of Microbiology, Health Canada, 1015 Arlington Street, Winnipeg, Manitoba, Canada, R3E 3R2, on Dec. 14, 2004, under Accession Number 141204-01. In accordance with 37 CFR 1.808, the depositors assure that all restrictions imposed on the availability to the public of the deposited materials will be irrevocably removed upon the granting of a patent. The deposit will be replaced if the depository cannot dispense viable samples.

Throughout the instant specification, hybridoma cell lines, as well as the isolated monoclonal antibodies which are produced therefrom, are alternatively referred to by their internal designation, AR40A746.2.3 or Depository Designation, IDAC 141204-01.

As used herein “antibody-ligand” includes a moiety which exhibits binding specificity for at least one epitope of the target antigen, and which may be an intact antibody molecule, antibody fragments, and any molecule having at least an antigen-binding region or portion thereof (i.e., the variable portion of an antibody molecule), e.g., an Fv molecule, Fab molecule, Fab′ molecule, F(ab′).sub.2 molecule, a bispecific antibody, a fusion protein, or any genetically engineered molecule which specifically recognizes and binds at least one epitope of the antigen bound by the isolated monoclonal antibody produced by the hybridoma cell line designated as IDAC 141204-01 (the IDAC 141204-01 antigen), a humanized antibody of the isolated monoclonal antibody produced by the hybridoma deposited with the IDAC as accession number 141204-01, a chimeric antibody of the isolated monoclonal antibody produced by the hybridoma deposited with the IDAC as accession number 141204-01 and antigen binding fragments.

As used herein “cancerous disease modifying antibodies” (CDMAB) refers to monoclonal antibodies which modify the cancerous disease process in a manner which is beneficial to the patient, for example by reducing tumor burden or prolonging survival of tumor bearing individuals, and antibody-ligands thereof.

A “CDMAB related binding agent”, in its broadest sense, is understood to include, but is not limited to, any form of human or non-human antibodies, antibody fragments, antibody ligands, or the like, which competitively bind to at least one CDMAB target epitope.

A “competitive binder” is understood to include any form of human or non-human antibodies, antibody fragments, antibody ligands, or the like which has binding affinity for at least one CDMAB target epitope.

As used herein “antigen-binding region” means a portion of the molecule which recognizes the target antigen.

As used herein “competitively inhibits” means being able to recognize and bind a determinant site to which the monoclonal antibody produced by the hybridoma cell line designated as IDAC 141204-01, (the IDAC 141204-01 antibody), a humanized antibody of the isolated monoclonal antibody produced by the hybridoma deposited with the IDAC as accession number 141204-01, a chimeric antibody of the isolated monoclonal antibody produced by the hybridoma deposited with the IDAC as accession number 141204-01, antigen binding fragments, or antibody ligands thereof, is directed using conventional reciprocal antibody competition assays. (Belanger L., Sylvestre C. and Dufour D. (1973), Enzyme linked immunoassay for alpha fetoprotein by competitive and sandwich procedures. Clinica Chimica Acta 48, 15).

As used herein “target antigen” is the IDAC 141204-01 antigen or portions thereof.

As used herein, an “immunoconjugate” means any molecule or CDMAB such as an antibody chemically or biologically linked to cytotoxins, radioactive agents, cytokines, interferons, target or reporter moieties, enzymes, toxins, anti-tumor drugs or therapeutic agents. The antibody or CDMAB may be linked to the cytotoxin, radioactive agent, cytokine, interferon, target or reporter moiety, enzyme, toxin, anti-tumor drug or therapeutic agent at any location along the molecule so long as it is able to bind its target. Examples of immunoconjugates include antibody toxin chemical conjugates and antibody-toxin fusion proteins.

Radioactive agents suitable for use as anti-tumor agents are known to those skilled in the art. For example, 131I or 211At is used. These isotopes are attached to the antibody using conventional techniques (e.g. Pedley et al., Br. J. Cancer 68, 69-73 (1993)). Alternatively, the anti-tumor agent which is attached to the antibody is an enzyme which activates a prodrug. A prodrug may be administered which will remain in its inactive form until it reaches the tumor site where it is converted to its cytotoxin form once the antibody complex is administered. In practice, the antibody-enzyme conjugate is administered to the patient and allowed to localize in the region of the tissue to be treated. The prodrug is then administered to the patient so that conversion to the cytotoxic drug occurs in the region of the tissue to be treated. Alternatively, the anti-tumor agent conjugated to the antibody is a cytokine such as interleukin-2 (IL-2), interleukin-4 (IL-4) or tumor necrosis factor alpha (TNF-α). The antibody targets the cytokine to the tumor so that the cytokine mediates damage to or destruction of the tumor without affecting other tissues. The cytokine is fused to the antibody at the DNA level using conventional recombinant DNA techniques. Interferons may also be used.

As used herein, a “fusion protein” means any chimeric protein wherein an antigen binding region is connected to a biologically active molecule, e.g., toxin, enzyme, fluorescent proteins, luminescent marker, polypeptide tag, cytokine, interferon, target or reporter moiety or protein drug.

The invention further contemplates CDMAB of the present invention to which target or reporter moieties are linked. Target moieties are first members of binding pairs. Anti-tumor agents, for example, are conjugated to second members of such pairs and are thereby directed to the site where the antigen-binding protein is bound. A common example of such a binding pair is avidin and biotin. In a preferred embodiment, biotin is conjugated to the target antigen of the CDMAB of the present invention, and thereby provides a target for an anti-tumor agent or other moiety which is conjugated to avidin or streptavidin. Alternatively, biotin or another such moiety is linked to the target antigen of the CDMAB of the present invention and used as a reporter, for example in a diagnostic system where a detectable signal-producing agent is conjugated to avidin or streptavidin.

Detectable signal-producing agents are useful in vivo and in vitro for diagnostic purposes. The signal producing agent produces a measurable signal which is detectable by external means, usually the measurement of electromagnetic radiation. For the most part, the signal producing agent is an enzyme or chromophore, or emits light by fluorescence, phosphorescence or chemiluminescence. Chromophores include dyes which absorb light in the ultraviolet or visible region, and can be substrates or degradation products of enzyme catalyzed reactions.

Moreover, included within the scope of the present invention is use of the present CDMAB in vivo and in vitro for investigative or diagnostic methods, which are well known in the art. In order to carry out the diagnostic methods as contemplated herein, the instant invention may further include kits, which contain CDMAB of the present invention. Such kits will be useful for identification of individuals at risk for certain type of cancers by detecting over-expression of the CDMAB's target antigen on cells of such individuals.

In order that the invention herein described may be more fully understood, the following description is set forth.

The present invention provides CDMAB (i.e., IDAC 141204-01 CDMAB, a humanized antibody of the isolated monoclonal antibody produced by the hybridoma deposited with the IDAC as accession number 141204-01, a chimeric antibody of the isolated monoclonal antibody produced by the hybridoma deposited with the IDAC as accession number 141204-01, antigen binding fragments, or antibody ligands thereof) which specifically recognize and bind the IDAC 141204-01 antigen.

The CDMAB of the isolated monoclonal antibody produced by the hybridoma deposited with the IDAC as accession number 141204-01 may be in any form as long as it has an antigen-binding region which competitively inhibits the immunospecific binding of the isolated monoclonal antibody produced by hybridoma IDAC 141204-01 to its target antigen. Thus, any recombinant proteins (e.g., fusion proteins wherein the antibody is combined with a second protein such as a lymphokine or a tumor inhibitory growth factor) having the same binding specificity as the IDAC 141204-01 antibody fall within the scope of this invention.

In one embodiment of the invention, the CDMAB is the IDAC 141204-01 antibody.

In other embodiments, the CDMAB is an antigen binding fragment which may be a Fv molecule (such as a single-chain Fv molecule), a Fab molecule, a Fab′ molecule, a F(ab′)2 molecule, a fusion protein, a bispecific antibody, a heteroantibody or any recombinant molecule having the antigen-binding region of the IDAC 141204-01 antibody. The CDMAB of the invention is directed to the epitope to which the IDAC 141204-01 monoclonal antibody is directed.

The CDMAB of the invention may be modified, i.e., by amino acid modifications within the molecule, so as to produce derivative molecules. Chemical modification may also be possible. Modification by direct mutation, methods of affinity maturation, phage display or chain shuffling may also be possible.

Affinity and specificity can be modified or improved by mutating CDR and/or phenylalanine tryptophan (FW) residues and screening for antigen binding sites having the desired characteristics (e.g., Yang et al., J. Mol. Biol., (1995) 254: 392-403). One way is to randomize individual residues or combinations of residues so that in a population of otherwise identical antigen binding sites, subsets of from two to twenty amino acids are found at particular positions. Alternatively, mutations can be induced over a range of residues by error prone PCR methods (e.g., Hawkins et al., J. Mol. Biol., (1992) 226: 889-96). In another example, phage display vectors containing heavy and light chain variable region genes can be propagated in mutator strains of E. coli (e.g., Low et al., J. Mol. Biol., (1996) 250: 359-68). These methods of mutagenesis are illustrative of the many methods known to one of skill in the art.

Another manner for increasing affinity of the antibodies of the present invention is to carry out chain shuffling, where the heavy or light chain are randomly paired with other heavy or light chains to prepare an antibody with higher affinity. The various CDRs of the antibodies may also be shuffled with the corresponding CDRs in other antibodies.

Derivative molecules would retain the functional property of the polypeptide, namely, the molecule having such substitutions will still permit the binding of the polypeptide to the IDAC 141204-01 antigen or portions thereof.

These amino acid substitutions include, but are not necessarily limited to, amino acid substitutions known in the art as “conservative”.

For example, it is a well-established principle of protein chemistry that certain amino acid substitutions, entitled “conservative amino acid substitutions,” can frequently be made in a protein without altering either the conformation or the function of the protein.

Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments.

### EXAMPLE 1

### In Vitro Binding on AML Stem Cells

AR40A746.2.3 monoclonal antibody was produced by culturing the hybridoma in CL-1000 flasks (BD Biosciences, Oakville, ON) with collections and reseeding occurring twice/week. Standard antibody purification procedures with Protein G Sepharose 4 Fast Flow (Amersham Biosciences, Baie d'Urfe, QC) were followed. It is within the scope of this invention to utilize monoclonal antibodies that are de-immunized, humanized, chimeric or murine.

Binding of AR40A746.2.3 to leukemic AML CD34+CD38− cells and normal CD34+CD38− cells was assessed by flow cytometry (FACS). One patient sample of AML and 3 samples of normal cells were tested. Normal CD34+CD38− cells were obtained from 2 samples of cord blood or 1 sample of normal adult bone marrow. Peripheral blood cells were collected from patients with newly diagnosed AML after obtaining informed consent according to procedures approved by the Research Ethics Board of the University Health Network (Toronto, ON). Individuals were diagnosed with AML according to the standards of the French-American-British classifications. Both human cord blood cells obtained from full-term deliveries and human adult bone marrow cells were obtained from consenting healthy donors according to procedures approved by the Research Ethics Boards of the University Health Network (Toronto, ON). Mononuclear blood cells from patient and normal donors were isolated by Ficoll Hypaque density gradient centrifugation and then were used right away or cryopreserved in fetal calf serum containing ten percent dimethyl sulfoxide. If cells were frozen, standard thawing procedures were followed before use.

For labeling CD34+CD38− leukemic cells, AML cells were stained with anti-CD34-PE-cyanine 5 (Beckman-Coulter, Mississauga, ON) and anti-CD38-PE (Becton-Dickinson, Oakville, ON) and were then analyzed using a FACScalibur (Becton-Dickinson, Oakville, ON). Quantities of CD9 on the cell surface were assessed with biotin-conjugated Arius antibody AR40A746.2.3. Cells labeled by AR40A746.2.3 were detected by adding allophycocyanin-conjugated streptavidin antibodies.

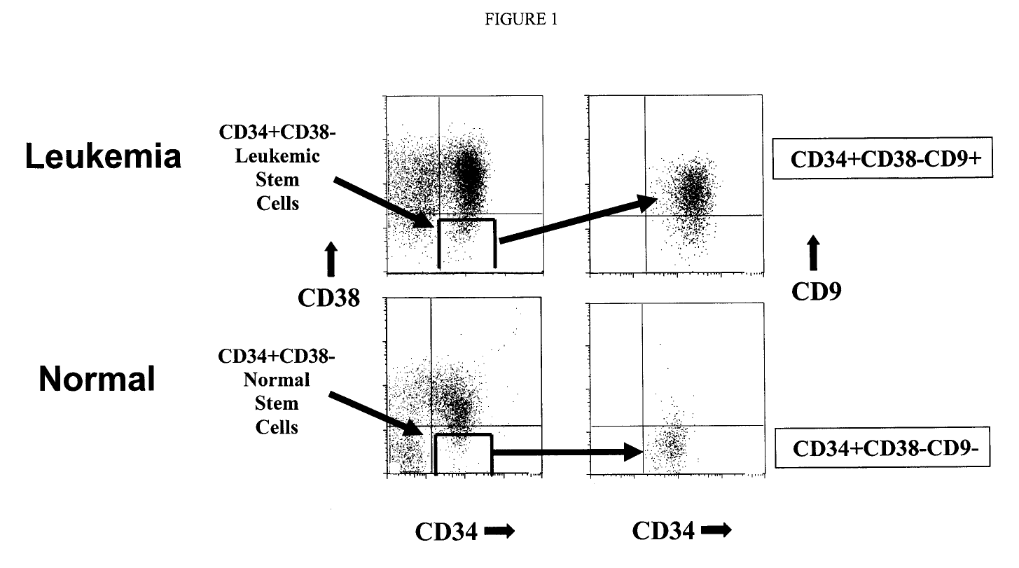

FIG. 1 presents representative profiles of AR40A746.2.3 antibodies binding to AML and normal cells. AR40A746.2.3 demonstrated binding to 85.8 percent of the CD34+CD38− cells from the AML patient sample. From the two normal cord blood and one normal bone marrow sample, CD9 expression was detected on 9.9 percent, 1.2 percent and 0 percent respectively. These data demonstrate that AR40A746.2.3 expression is differentially expressed on leukemic versus normal CD34+/CD38− cells, which in turn demonstrates that CD9 expression is up regulated in cancer stem cells.

### EXAMPLE 2

### In Vitro Binding on AML, ALL and CML Stem Cells

Example 1 demonstrates that CD9 expression is up regulated in CD34+/CD38− stem cells from an AML patient sample in comparison to several normal samples. To determine whether CD9 expression is up regulated in other types of leukemia besides AML, AR40A746.2.3 was tested by FACS against several samples of AML, ALL and CML and compared to normal bone marrow and cord blood samples. The samples were obtained as outlined in Example 1. The FACS procedure was the same as the one outlined above. FACS was performed on 7 AML, 3 ALL and 1 CML patient and was compared to 2 normal bone marrow and 2 normal cord blood samples.

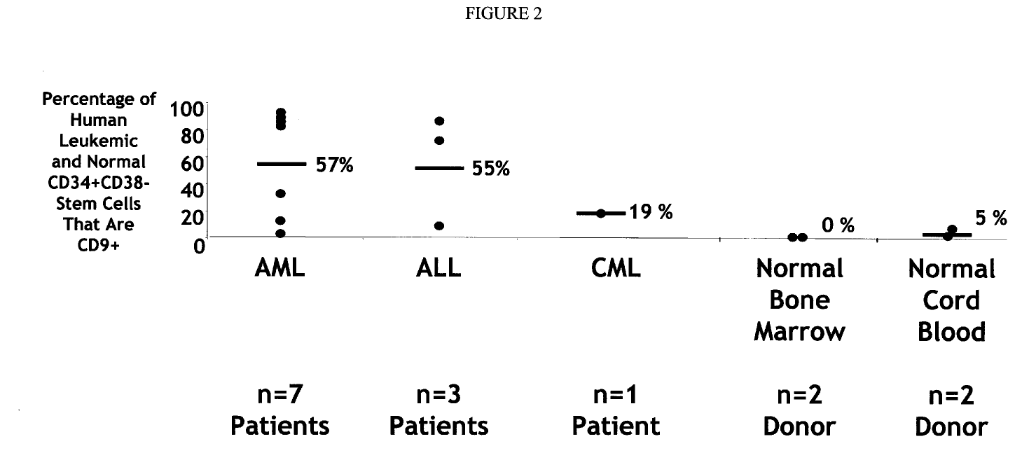

FIG. 2 presents the average percentage of CD34+CD38− cells that demonstrated CD9 expression across the various patient and donor (normal) samples. The average patient sample percentages were 57 percent, 55 percent and 19 percent for AML, ALL and CML respectively. For the normal bone marrow and normal cord blood samples, the average percentages were 0 and 5 percent respectively. Four out of the 7 AML patient samples had greater than 80 percent of the CD34+CD38− stem cells also exhibit CD9 expression. These data further confirm that CD9 expression is increased in AML patient stem cells. In addition, it demonstrates that CD9 expression is up regulated in other leukemic stem cells, including ALL and CML. Together, these data demonstrate that leukemic stem cells are CD34+CD38-CD9+.

### EXAMPLE 3

In Vivo Experiment with Human AML Cells

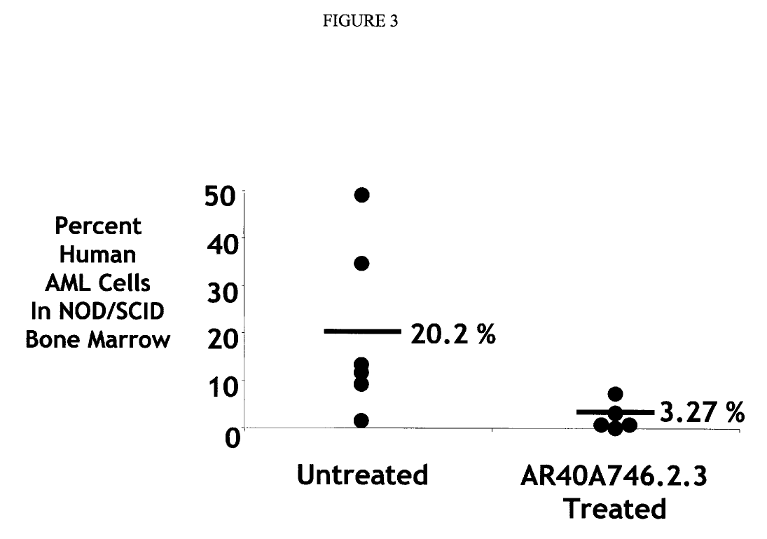

Examples 1 and 2 demonstrate that CD9 is over expressed on CD34+CD38− cancer stem cells. To determine the utility of the anti-CD9 AR40A746.2.3 antibody for the treatment of leukemia, AR40A746.2.3 was tested in a human AML mouse model. With reference to FIG. 3, 8-12 week old female NOD/SCID mice were sublethally irradiated with 3.6 Gy before being injected with 1 million human AML cancer cells in 200-500 microliters PBS solution injected intravenously in the tail vein. The mice were randomly divided into 2 treatment groups. Some mice were lost before treatment due to the sublethal dose of radiation. On day 10 after injection, 20 mg/kg of AR40A746.2.3 test antibody or buffer control was administered intraperitoneally to each cohort in a volume of 300 microliters after dilution from the stock concentration with a diluent that contained 2.7 mM KCl, 1 mM KH2PO4, 137 mM NaCl and 20 mM Na2HPO4. Antibody was injected on day 10 after the injection of AML cells in order to allow for the grafting of the cells into the bone marrow of the mice. The antibody and control samples were then administered twice per week for four weeks. Mice were sacrificed on day 40 after cell implantation and the bone marrow was flushed to harvest the cells. Human AML cells were detected by FACS with the use of anti-human CD45 antibody, anti-CD45-phycoerythrin (Becton-Dickinson, Oakville, ON). Since CD45 is a pan marker of human hematopoietic cells, CD45 levels were measured as an indicator of the number of human AML cells in the murine bone marrow. Mouse IgG-phycoerythrin (Becton-Dickinson, Oakville, ON) was used as the isotypic control. A FACScalibur (Becton-Dickinson, Oakville, ON) was used for the flow cytometry analysis. Animal experiments were performed in accordance with institutional guidelines approved by the University Health Network/Princess Margaret Hospital Animal Care Committee (Toronto, ON).

AR40A746.2.3 treatment reduced the percentage of human AML cells in the in vivo murine model of human AML. Treatment with Arius antibody AR40A746.2.3 reduced the average percentage of human AML cells from 20.2 percent in the untreated mice to 3.3 percent in the AR40A746.2.3-treated group (p=0.07, t-test)(FIG. 3).

There were no clinical signs of toxicity throughout the study. Body weight measured at regular intervals was a surrogate for well-being and failure to thrive. There were no significant differences in body weight between the groups at the end of the treatment period. In summary, AR40A746.2.3 was well-tolerated and decreased the leukemic outgrowth in this human AML murine model.

### EXAMPLE 4

In Vivo Experiment with Human AML Cells

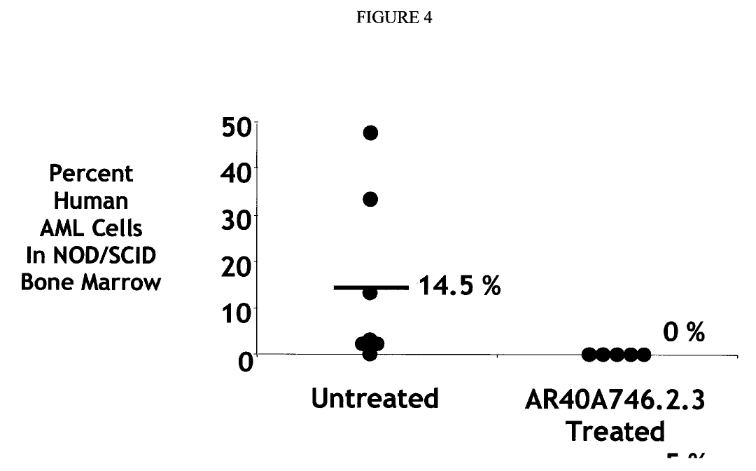

Example 3 demonstrates that treatment with anti-CD9 antibody AR40A746.2.3 reduces the number of AML cells in the bone marrow in comparison to untreated mice. To determine whether AR40A746.2.3 is inhibiting the human AML cancer stem cells that led to the outgrowth, secondary transplantation into new NOD/SCID mice was done. Secondary transplantation can be used as a test of the self-renewing and pluripotency of normal and cancer stem cells (Hope et al., Nat. Immunol. 5:738-743 2004). With reference to FIG. 4, bone marrow from the primary recipient mice (the mice used in Example 3) was harvested and transplanted into secondary recipients (in the same manner as outlined in Example 3). After 8 weeks, the percent of human AML cells in the NOD/SCID bone marrow was determined (as outlined in Example 3).

AR40A746.2.3 reduced the percentage of human AML cells in the secondary recipients. Treatment with Arius antibody AR40A746.2.3 significantly reduced the average percentage of human AML cells from 14.5 percent in the untreated mice to 3.3 percent in the AR40A746.2.3-treated group (p<0.05, t-test)(FIG. 4).

In summary, AR40A746.2.3 decreased the leukemic outgrowth in both the primary and secondary recipients of this human AML murine model. This data also demonstrates that treatment with anti-CD9 antibody AR40A746.2.3 inhibits AML cancer stem cells since AML outgrowth was abolished in secondary recipients. Taken together, this data demonstrates that leukemic stem cells are CD34+/CD38−/CD9+ and that treatment with anti-CD9 antibody AR40A746.2.3 substantially reduces the number of cancer stem cells suggesting pharmacologic and pharmaceutical benefits of this antibody for therapy in other mammals including man. In toto, this data demonstrates that the AR40A746.2.3 antigen is a cancer stem cell associated antigen and is expressed on human cancer stem cells, and is a pathologically relevant cancer target.

The method of treatment described herein, particularly for cancers, may also be carried out with administration of other antibodies or chemotherapeutic agents. For example, an antibody against EGFR, such as ERBITUX® (cetuximab), may also be administered, particularly when treating colon cancer. ERBITUX® has also been shown to be effective for treatment of psoriasis. Other antibodies for combination use include Herceptin® (trastuzumab) particularly when treating breast cancer, AVASTIN® particularly when treating colon cancer and particularly when treating non-small cell lung cancer. The administration of the antibody of the present invention with other antibodies/chemotherapeutic agents may occur simultaneously, or separately, via the same or different route.

The chemotherapeutic agent/other antibody regimens utilized include any regimen believed to be optimally suitable for the treatment of the patient's condition. Different malignancies can require use of specific anti-tumor antibodies and specific chemotherapeutic agents, which will be determined on a patient to patient basis. In a preferred embodiment of the invention, chemotherapy is administered concurrently with or, more preferably, subsequent to antibody therapy. It should be emphasized, however, that the present invention is not limited to any particular method or route of administration.

The preponderance of evidence shows that AR40A746.2.3 mediates anti-cancer effects and prolongs survival through ligation of epitopes present on CD9. It has been shown (as disclosed in Example 13) that AR40A746.2.3 antibodies can be used to immunoprecipitate the cognate antigen from expressing cells such as BxPC-3 cells. Further it could be shown that AR40A746.2.3, chimeric AR40A746.2.3 or humanized variants can be used in the detection of cells and/or tissues which express a CD9 antigenic moiety which specifically binds thereto, utilizing techniques illustrated by, but not limited to FACS, cell ELISA or IHC.

As with the AR40A746.2.3 antibody, other anti-CD9 antibodies could be used to immunoprecipitate and isolate other forms of the CD9 antigen, and the antigen can also be used to inhibit the binding of those antibodies to the cells or tissues that express the antigen using the same types of assays.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification.

One skilled in the art will readily appreciate that the present invention is well adapted to carry out the objects and obtain the ends and advantages mentioned, as well as those inherent therein. Any oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method for the treatment of a human hematologic malignancy in a mammal, wherein said human hematologic malignancy expresses at least one epitope of an antigen which specifically binds to the isolated monoclonal antibody produced by the hybridoma deposited with the IDAC as accession number 141204-01 or a CDMAB thereof, which CDMAB is characterized by an ability to competitively inhibit binding of said isolated monoclonal antibody to its target antigen, comprising administering to said mammal said monoclonal antibody or CDMAB thereof in an amount effective to result in a reduction of said mammal's tumor burden.

2. The method of claim 1 wherein the hematologic malignancy is selected from the group consisting of Acute Myeloid Leukemia (AML), Chronic Lymphocyte Leukemia (CLL), Chronic Myeloid Leukemia (CML) and Multiple Myeloma.

3. The method of claim 1 wherein said isolated monoclonal antibody is conjugated to a cytotoxic moiety.

4. The method of claim 3 wherein said cytotoxic moiety is a radioactive isotope.

5. The method of claim 1 wherein said isolated monoclonal antibody is a humanized antibody of the isolated monoclonal antibody produced by the hybridoma deposited with the IDAC as accession number 141204-01 or an antigen binding fragment produced from said humanized antibody.

6. The method of claim 1 wherein said isolated monoclonal antibody is a chimeric antibody of the isolated monoclonal antibody produced by the hybridoma deposited with the IDAC as accession number 141204-01 or an antigen binding fragment produced from said chimeric antibody.

7. The use of monoclonal antibodies for the treatment of a human hematologic malignancy in a mammal, wherein said hematologic malignancy expresses at least one epitope of an antigen which specifically binds to the isolated monoclonal antibody produced by the hybridoma deposited with the IDAC as accession number 141204-01 or a CDMAB thereof, which CDMAB is characterized by an ability to competitively inhibit binding of said isolated monoclonal antibody to its target antigen, comprising administering to said mammal said monoclonal antibody or CDMAB thereof in an amount effective to result in a reduction of said mammal's tumor burden.

8. The use of claim 7 wherein said isolated monoclonal antibody is conjugated to a cytotoxic moiety.

9. The use of claim 8 wherein said cytotoxic moiety is a radioactive isotope.

10. The use of claim 7 wherein said isolated monoclonal antibody is a humanized antibody of the isolated monoclonal antibody produced by the hybridoma deposited with the IDAC as accession number 141204-01.

11. The use of claim 7 wherein said isolated monoclonal antibody is a chimeric antibody of the isolated monoclonal antibody produced by the hybridoma deposited with the IDAC as accession number 141204-01.

12. A method for the treatment of a human hematologic malignancy in a mammal, wherein said hematologic malignancy expresses at least one epitope of an antigen which specifically binds to the isolated monoclonal antibody produced by the hybridoma deposited with the IDAC as accession number 141204-01 or a CDMAB thereof, which CDMAB is characterized by an ability to competitively inhibit binding of said isolated monoclonal antibody to its target antigen, comprising administering to said mammal said monoclonal antibody or CDMAB thereof; in conjunction with at least one chemotherapeutic agent in an amount effective to result in a reduction of said mammal's tumor burden.

13. The method of claim 12 wherein said isolated monoclonal antibody or CDMAB is conjugated to said chemotherapeutic agent.

14. The method of claim 13 wherein said chemotherapeutic agent is a cytotoxic moiety.

15. The method of claim 13 wherein said cytotoxic moiety is a radioactive isotope.

16. The method of claim 12 wherein said isolated monoclonal antibody is a humanized antibody of the isolated monoclonal antibody produced by the hybridoma deposited with the IDAC as accession number 141204-01.

17. The method of claim 12 wherein said isolated monoclonal antibody is a chimeric antibody of the isolated monoclonal antibody produced by the hybridoma deposited with the IDAC as accession number 141204-01.

18. A composition effective for the treatment of a human hematologic malignancy comprising in combination:
an isolated monoclonal antibody or CDMAB produced by a hybridoma deposited with the IDAC as accession number 141204-01; and
a requisite amount of a pharmacologically acceptable carrier;
wherein said composition is effective for treating said human hematologic malignancy.

19. The composition of claim 18, further including a conjugate of said isolated monoclonal antibody or an antigen binding fragment thereof with a member selected from the group consisting of cytotoxic moieties, enzymes, radioactive compounds, cytokines, interferons, target or reporter moieties and hematogenous cells;

20. An assay kit for detecting the presence of a human hematologic malignancy, wherein said human hematologic malignancy expresses at least one epitope of an antigen which specifically binds to the isolated monoclonal antibody produced by the hybridoma deposited with the IDAC as accession number 141204-01 or a CDMAB thereof, which CDMAB is characterized by an ability to competitively inhibit binding of said isolated monoclonal antibody to its target antigen, the kit comprising the isolated monoclonal antibody produced by the hybridoma deposited with the IDAC as accession number 141204-01 or a CDMAB thereof, and means for detecting whether the monoclonal antibody, or a CDMAB thereof, is bound to a polypeptide whose presence, at a particular cut-off level, is diagnostic of said presence of said human hematologic malignancy.
